(19) United States
(12) Patent Application Publication
(10) Pub. No.: US 2022/0002655 A1
(43) Pub. Date: Jan. 6, 2022

(54) MICROFLUIDIC IN VITRO MODEL FOR ELUCIDATING THE MOLECULAR EFFECTS OF SIMULATED DIETARY REGIMENS ON GUT MICROBIOTA AND HOST CELLS

(71) Applicant: Universite Du Luxembourg, Esch-sur-Alzette (LU)

(72) Inventors: Paul Wilmes, Bettembourg (LU); Kacy Greenhalgh, Luxembourg (LU)

(21) Appl. No.: 17/294,587

(22) PCT Filed: Nov. 15, 2019
(86) PCT No.: PCT/EP2019/081424

§ 371 Date: May 17, 2021

(30) Foreign Application Priority Data
Nov. 16, 2018 (EP) 18206858.5
Nov. 19, 2018 (LU) 101002
Nov. 21, 2018 (LU) 101006

(51) Int. Cl. C12M 1/34; C12M 3/06; C12M 1/00; G01N 33/50

(52) U.S. Cl. CPC C12M 41/46; C12M 23/16; G01N 33/502; G01N 33/5082; C12M 29/04

(57) ABSTRACT

A microfluidic cell culture device for performing dietary compounds—host microbiota cells molecular interactions, the microfluidic cells culture device comprising two or more channels, wherein at least two adjacent channels are cell culture channels separated by a permeable or semi permeable membrane adapted to prevent passage of cells thereacross, a first channel of the at least two adjacent channels supporting a culture of microbiota cells of a host and a second of the at least two channels supporting at least one probiotics culture and being perfused with a medium of dietary compound.

### CROSS-REFERENCE TO RELATED APPLICATIONS

The present invention is the US national stage under 35 U.S.C. § 371 of International Application No. PCT/EP2019/081424 which was filed on Nov. 15, 2019, and which claims the priority of application EP 18206858.5 filed on Nov. 16, 2018, LU 101002 filed on Nov. 19, 2018, and LU 101006 filed on Nov. 21, 2018, the content of which (text, drawings and claims) are incorporated here by reference in its entirety.

### FIELD

The invention relates to the study of diet—gut microbiota interactions in relation to human health. In particular, this invention is directed to the use of a microfluidic in vitro system for investigating simulated dietary regimens and more particularly the combinatorial effects of prebiotics together with probiotics on gut microbiota cells.

### BACKGROUND

The human gut microbiome is increasingly recognized as playing a major role in human health and disease (Pflughoeft and Versalovic, 2012). Modulation of the gut microbiome using prebiotics, that are non-digestible polysaccharides such as dietary fiber, which promote the growth of beneficial microorganisms in the host, together with probiotics, that are live microorganisms which when administered in adequate amounts, confer health benefits to the host, or combinations thereof (synbiotics), is regarded as a means to prevent microbiome-linked diseases, such as colorectal cancer (CRC) (Rafter et al., 2007; Raman et al., 2013) to act as a possible supportive therapeutic options (DiMarco-Crook and Xiao, 2015; Ho et al., 2018). However, although microbiome-modulating therapeutics hold great promise (Valencia et al., 2017), the combination of prebiotics and probiotics are not formally integrated in current treatment plans (Caccialanza et al., 2016).

Due to the limitations of existing methodologies, in particular the lack of means to study the molecular effects of dietmicrobiome-host interactions (Read and Holmes, 2017), limited evidence exists on the therapeutic benefits of prebiotics and probiotics to prevent or treat microbiome-linked diseases.

Concerning the CRC, limited number of studies have focused on inflammatory and proliferative signatures but these have not assessed the linked changes in gene expression or metabolism (Ho et al., 2018; Le Leu et al., 2005).

In this context, elucidating the mechanisms of action of synbiotic regimens (i.e., prebiotics+probiotics) may in particular prove valuable to improve the efficacy of current anti-cancer treatments.

As CRC is mostly driven by environmental factors, such as diet and a broad range of mutations (Blot and Tarone, 2015; Rothenberg, 2015; Armaghany et al., 2012), it is challenging to recapitulate the complexity of the disease using only one specific animal model, in particular the widely murine model, since this model presents differences, among others, in diet, gut topology, genetic background and microbiome composition rendering this model questionable for investigating mechanisms underlying human host-microbiome interactions (Fritz et al., 2013; Hildebrand et al., 2013).

Recent studies (Bein et al., 2018; Wilmes et al., 2018) showed that in vitro gut-on-chip models allow recapitulation of human gastrointestinal physiology and thereby allow the probing of molecular exchanges between microbial and human cells and their repercussions in a representative manner.

### SUMMARY

The invention has for technical problem to provide a solution to at least one of the drawbacks of the above prior arts. More particularly, the present invention has for technical problem to provide an efficient solution that allows the investigation of molecular interactions between diet-host microbiota cells.

For this purpose, the invention is directed to the use of a microfluidic cell culture device for performing dietary compounds—host microbiota cells molecular interactions, said microfluidic cells culture device comprising two or more channels, wherein at least two adjacent channels are cell culture channels separated by a permeable or semi permeable membrane adapted to prevent passage of cells thereacross, a first channel of the at least two adjacent channels supporting a culture of microbiota cells of a host and a second of the at least two channels supporting at least one probiotics culture and being perfused with a medium of dietary compounds.

Advantageously, the microfluidic cell culture device is constructed in layers, with individual layers for each channel and for each membrane.

Advantageously, the adjacent channels take the form of a paired helix.

Advantageously, the microfuidic cell culture device further comprises a third channel, the third channel being separated from said first channel by a semipermeable membrane, the third channel being configured to carry nutrients to said first channel. Advantageously, this third channel is a perfusion channel.

Advantageously, the second of the at least two channels comprises one or more dwell chambers.

According to an exemplary embodiment, the microfluidic cell culture device is for investigating the combinatorial combinations of dietary compounds and probiotics, resulting in the production by probiotics of molecular compounds enabling to modulate at the molecular level the host microbiota cells.

According to an exemplary embodiment, dietary compounds comprise prebiotics.

According to an exemplary embodiment, prebiotics comprise dietary fibers, carbohydrates selected from the group consisting of disaccharides, oligosaccharides, polysaccharides, and/or mixtures thereof.

According to an exemplary embodiment, dietary fibers are selected from non-starch derived indigestible polysaccharides, galacto-oligosaccharides and fructo-oligosaccharides, and/or mixture thereof.

According to an exemplary embodiment, the probiotics culture comprises at least one bacteria species.

According to an exemplary embodiment, the probiotics culture comprises at least one bacteria species from gut microbiome.

According to an exemplary embodiment, the probiotics culture comprises at least one bacteria species selected from Lactobacillus species.

According to an exemplary embodiment, Lactobacillus species is selected from L. rhamnosus, L. acidophilus, L. delbrueckii, L. helveticus, L. casei, L. curvatus, L. plantarum, L. sakei, L. brevis, L. bruchneri, L. fermentum, L. reuteri.

According to an exemplary embodiment, the culture of microbiota cells is from a mammalian host.

According to an exemplary embodiment, the culture of microbiota cells is from a human host.

According to an exemplary embodiment, the molecular compounds secreted by the probiotics are dietary compound-dependent.

According to an exemplary embodiment, the molecular compounds secreted by the probiotics are probiotics dependent.

According to an exemplary embodiment, the molecular compounds secreted by the probiotics comprise organic and short chain fatty acids.

According to an exemplary embodiment, the molecular compounds secreted by the probiotics comprise lactate, formate, acetate.

The invention is also directed to synbiotic regimens obtained by the use of the microfluidic cells culture device according to the invention.

According to an exemplary embodiment, synbiotic regimens are for use in treating and/or preventing human colorectal cancer cells.

According to an exemplary embodiment, synbiotic regimens are for use in treating and/or preventing human gut microbiome-linked diseases.

According to an exemplary embodiment, synbiotic regimens are for use in treating and/or preventing inflammatory diseases of gut.

According to an exemplary embodiment, synbiotic regimens are for use as adjuvant in combination with anti-cancer drug-treatments.

According to an exemplary embodiment, synbiotic regimens are for use as dietary supplement in combination with anti-cancer drug treatments.

According to an exemplary embodiment, symbiotic regimens are for use as a pharmaceutical composition.

According to an exemplary embodiment, synbiotic regimens have the form of a liquid, a powder, a granulate, a paste, a bare, an effervescent tablet, a tablet, a capsule, a lozenge, a fast melting tablet or wafer, a substance tablet or a spray.

The invention is also directed to a method for performing dietary compounds—host microbiota cells molecular interactions comprising (i) providing a microfluidic device comprising two or more channels, at least two adjacent of the channels are cell culture channels separated by a permeable or semi permeable membrane adapted to prevent passage of cells thereacross; (ii) populating a first channel of the channels with a culture of gut cells from a host, the gut cells being selected from cells making up the wall in at least one of the small intestine and colon, for example gastrointestinal tract epithelial cells; (iii) passing a probiotics culture comprising at least one bacteria species into a second channel of the channels; (iv) perfusing through the second channel a medium of dietary compounds comprising prebiotics with dietary fiber; (v) monitoring the interactions between the gut cells, prebiotics and probiotics by means allowing the interrogation of molecular interactions by molecular techniques comprising imaging and/or spectroscopic techniques and/or one or more genomics, proteomics, metabolics, transcriptomics, or other molecular analysis techniques, and/or any combination thereof; (vi) isolating molecular compounds produced by probiotics.

According to an exemplary embodiment, the culture of gut cells is from mammalian, human or insect.

According to an exemplary embodiment, the culture of gut cells populating the first channel in step (ii) are from persons having microbiome-linked diseases.

According to an exemplary embodiment, the culture of gut cells populating the first channel in step (ii) is from persons having a colorectal cancer.

According to an exemplary embodiment, the method for performing dietary compounds—host microbiota cells molecular interactions —further comprises a step (vi) of selecting combinations of probiotics and prebiotics with dietary fibers for which the molecular analyses of step (v) show downregulation of genes involved in pro-carcinogenic pathways and drug resistance, and/or reduced levels of oncometabolites.

According to an exemplary embodiment, selected combinations of probiotics and prebiotics with fibers form symbiotic regimens.

The present invention is particularly interesting in that it enables to investigate molecular interactions driving dietary—host microbiota cells. This invention is all the more interesting that it permits to determine the potential combinatorial action of prebiotics together with probiotics on the gut cells of a host. In contrast to individual prebiotic or probiotic regimen, the symbiotic regimens cause downregulation of genes involved in procarcinogenic pathways and drug resistance, and result in reduced levels of the oncometabolite lactate. Molecular compounds synthesised by the probiotics during symbiotic regimens attenuate self-renewal capacity in primary CRC-derived cells, a cellular hallmark of tumour progression and disease dissemination. Actually, this invention is also interesting in that it provides mechanistic support regarding the potential of integrating synbiotic regimens in the context of therapeutic regimens for CRC. Such integrative in vitro and in silico modelling could be used to develop personalized treatments, including dietary guidelines and probiotic supplementation for human CRC patients.

### DRAWINGS

FIG. 1A shows the conceptual diagram of a in vitro human cells-microbe gut model HuMiX, in accordance with various embodiments of the invention.

FIG. 1B depicts the composition of two distinct dietary regimens, a HF regimen consisted of a medium high in starch and of dietary fibers (prebiotic), in accordance with various embodiments of the invention.

FIG. 2A presents the relative intracellular lactate concentrations in human Caco-2 cells after co-culture with HF regimen (prebiotic) versus REF regimen, in accordance with various embodiments of the invention.

FIG. 2B shows relative gene expression of lactate importer MCT1 and exporter MCT4 in Caco-2 cells after co-culture with the HF regimen (prebiotic) versus the REF medium, in accordance with various embodiments of the invention.

FIG. 3 shows in vitro glycolysis-related genes differentially expressed in HF-exposed cells compared to REF-exposed cells, in accordance with various embodiments of the invention.

FIGS. 4A and 4B show Caco-2 cells count in million and Caco-2 cells viability, respectively (HF medium (prebiotic); REF medium; HF (prebiotic)+LGG (probiotic); REF medium+LGG (probiotic)), in accordance with various embodiments of the invention.

FIG. 5A presents the global expression profiles of Caco-2 cells grown under different conditions (Caco-2 in HF medium (prebiotic); Caco-2 in REF medium; Caco-2 in HF (prebiotic)+LGG (probiotic); Caco-2 in REF medium+LGG (probiotic)), in accordance with various embodiments of the invention.

FIG. 5B shows the pathway enrichment of Caco-2 cells. Data are shown as the mean±SEM from three REF-exposed and four HF-exposed independent HuMiX experiments, in accordance with various embodiments of the invention.

FIG. 5C shows the relative expression of differentially expressed genes in Caco-2 cells after exposure to the HF regimen (prebiotic) or REF medium, in accordance with various embodiments of the invention.

FIG. 6A and FIG. 6B show LGG viability and LGG count, respectively (LGG+HF regimen (prebiotic); LGG+REF medium)), in accordance with various embodiments of the invention.

FIG. 7A presents the global expression profiles of LGG (probiotic) grown under HF (prebiotic) dietary regimens and co-culture with Caco-2 cells in HuMiX, in accordance with various embodiments of the invention.

FIG. 7B shows measurement of organic and short chain fatty acids secretion products by LGG (probiotic) grown in the presence of HF regimen (prebiotic) or REF medium, in accordance with various embodiments of the invention.

FIG. 8 illustrates the relative abundances of in vitro intracellular metabolites in Caco-2 cells and LGG after co-culture in HuMiX. (value based on three independent experiments; Colors indicate sample type and stronger shades indicate increased relative abundance), in accordance with various embodiments of the invention.

FIG. 9 shows symbiotic regimen (HF regimen (prebiotic)+LGG (probiotic)) which causes down regulation of CRC-associated genes and pathways in human Caco-2 cells. FIG. 9A presents enrichment pathway analysis of Caco-2 cells when exposed to HF regimen (prebiotic)+LGG (probiotic). FIG. 9B depicts the relative expression of differentially expressed ABC transporter genes in Caco-2 cells (in HF medium (prebiotic); REF medium; HF (prebiotic)+LGG (probiotic); REF medium+LGG (probiotic)), in accordance with various embodiments of the invention.

FIG. 10 shows metabolic products produced by LGG (probiotic) under different dietary regimens that differentially impact CRC cell growth Caco-2 cells and primary T-6 cells)—Three independent experiments). FIG. 10A shows effect of individual exposures to acetate, lactate and formate (10 mM) on CRC self-renewal capacity. FIG. 10B shows effect of exposure to the diet-dependent cocktail of molecules secreted by LGG (probiotic) on human CRC cells self-renewal capacity (SCFAs: short chain fatty acids), in accordance with various embodiments of the invention.

### DETAILED DESCRIPTION

The following discloses an embodiment merely exemplarily representative of the invention which may be embodied in various forms.

Establishment of an in vitro model system to study the interactions between dietary fiber including prebiotics, probiotics and the human host:

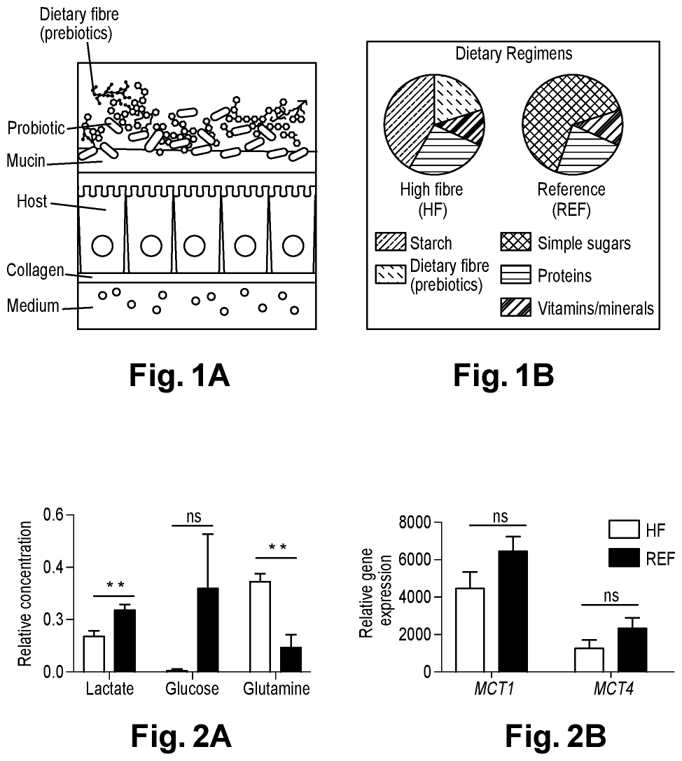

In this exemplary embodiment, the used microfluidic cell culture device is the HuMix model (Shah et al., 2016). In this exemplary case, this system comprises actually two adjacent cell chambers separated by a permeable or semi permeable membrane adapted to prevent passage of cells theracross, and a third chamber or bottom chamber (FIG. 1A). This system has been used to allow the exposure of a culture of human epithelial Caco-2 cells to dietary compounds and live Lactobacillus rhamnosus cells (LGG: probiotic). In parallel, two simulated dietary regimens simulating two groups of dietary compounds were used (FIG. 1B). The first dietary regimen was a prebiotic regimen which consists of a medium high in starch and dietary fiber including prebiotics. The second dietary regimen was a reference regimen (REF), it contains neither prebiotics or dietary fiber, nor starch. This REF medium is actually a human cell culture medium providing the basic requirements for culture of both human Coca-2 cells and LGG (Shah et al., 2016). The probiotics were cultured in the presence of the simulated dietary regimens in the top chamber separated via a nanoporous membrane from the middle chamber that houses Caco-2 cells. In this exemplary embodiment, the bottom chamber, is also separated from the middle chamber via a microporous membrane and contains cell culture medium which mediates the transport of nutrients to human cells basal surface. The dietary regimens are perfused chamber into the top chamber containing LGG. In the two parallel experiments, the HuMiX system allowed actually the exposure of human cells to dietary compounds of HF regimen and REF regimen and LGG via the apical interface, thereby mimicking the in vivo physiology and enabling the study of diet-host-microbe molecular interactions.

Interestingly, using a transwell system set up, no growth of human Caco-2 cells in the presence of the HF regimen could be observed while in contrast and using the in vitro microfluidic cell culture system described above, human Caco-2 cells were viable.

### A Simulated HF Regimen Alone Affects Energy Metabolism in Caco-2 Cells:

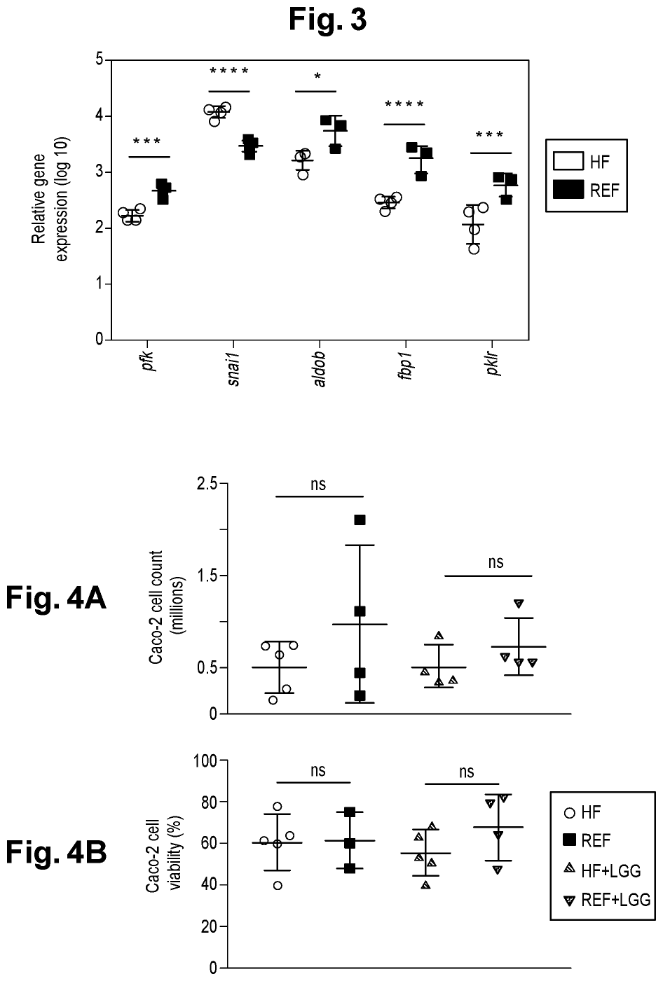

The effects of the HF regimen on the metabolism of human Caco-2 cells were then investigated. In vitro, the intracellular lactate concentrations in Caco-2 cells were significantly reduced (P=2.63×10⁻³) in the presence of the HF regimen in comparison to the REF condition (FIG. 2A).

In contrast, no significant changes in the expression of lactate transporters were observed in Caco-2 cells when exposed to the HF regimen in comparison to the REF medium (FIG. 2B). Additionally, the concentration of intracellular glucose was decreased, while the concentration of glutamine was significantly increased (P=2.43×10⁻³) in HF-exposed cells compared to REF-exposed cells (FIG. 2A).

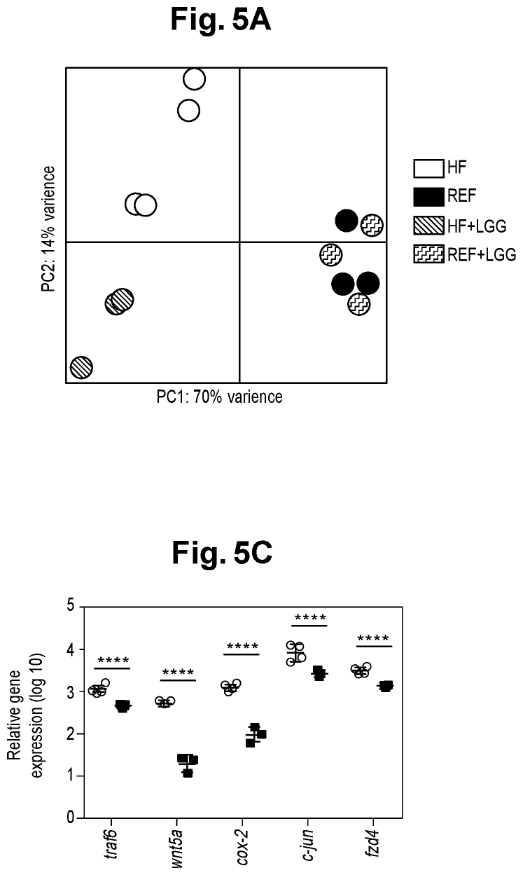

Expression of several glycolytic enzymes was found to be reduced in Caco-2 cells after co-culture with the simulated HF regimen (FIG. 3), including phosphofructokinase (pfk), aldolase B (aldob), fructo-1,6biphosphatase (fbpl) and pyruvate kinase isozymes R/L (PKLR) (FIG. 3). The snail family transcriptional repressor 1 (snail), which regulates glycolysis by inhibiting pfk expression (Kim et al., 2017), was increased (FIG. 3).

### A HF-Regimen Alone Activates Several Oncogenes and Proinflammatory Pathways in Caco-2 Cells:

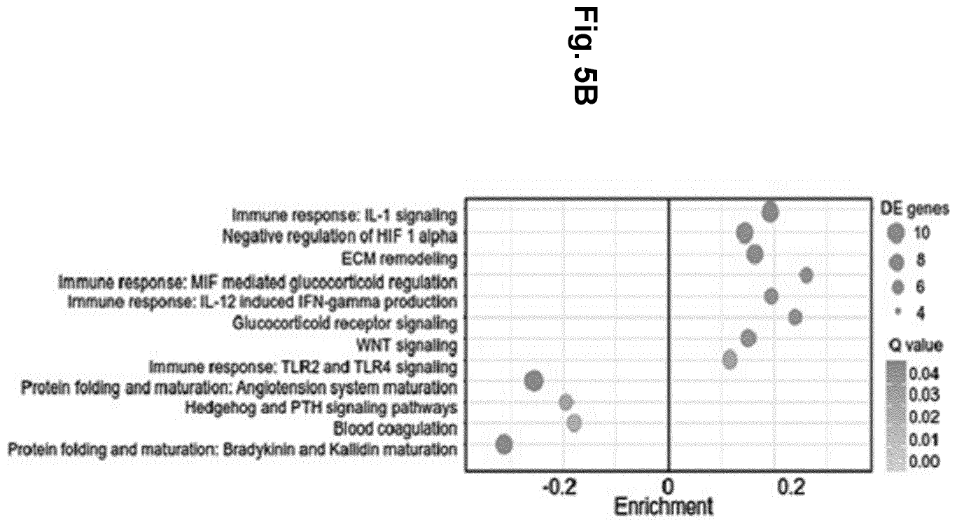

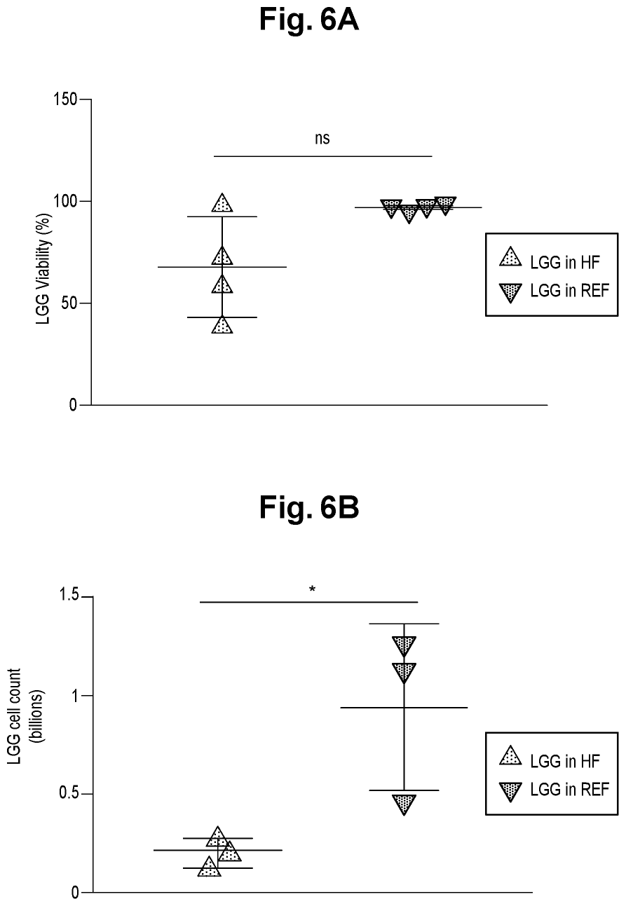

The effect of the HF regimen on human Caco-2 cell proliferation and viability has been evaluated. Even though growth and viability of Caco-2 cells in HuMiX were comparable between the simulated dietary regimens (FIG. 4A and FIG. 4B), the regimens had a pronounced effect on the global transcriptome profile of Caco-2 cells (FIG. 5A).

Pathway enrichment analysis showed that pathways, responsible for regulating inflammatory responses in CRC (Voronov and Apte, 2015; Wang and Dubois, 2010), e.g., IL-1 signaling, were significantly enriched in Caco-2 cells when exposed to the HF regimen (FIG. 5B). Within this pathway, the IL-1 receptor 1 (il-1r1), as well as its downstream target, TNF receptor associated factor (traf6), were significantly upregulated (FIG. 5C). Additional downstream target genes of the IL-1 signaling cascade which were significantly upregulated in Caco-2 cells included Cyclooxygenase-2 (cox-2) and c-jun (FIG. 5C).

The HF regimen also led to the upregulation of genes in the wingless/integrated (WNT) pathway (FIG. 5B) and increased the expression of the WNT ligand wnt5a as well as downstream targets such as snail and Frizzled-4 (fzd4; FIG. 5C), which are known to be involved in CRC progression and drug resistance (Chikazawa et al., 2010; Guo et al., 2016; Voronov and Apte, 2015; Zhan et al., 2017).

### A HF Regimen Affects Gene Expression and Metabolism of a Probiotic:

The effect of the HF regimen on LGG growth and viability has been evaluated.

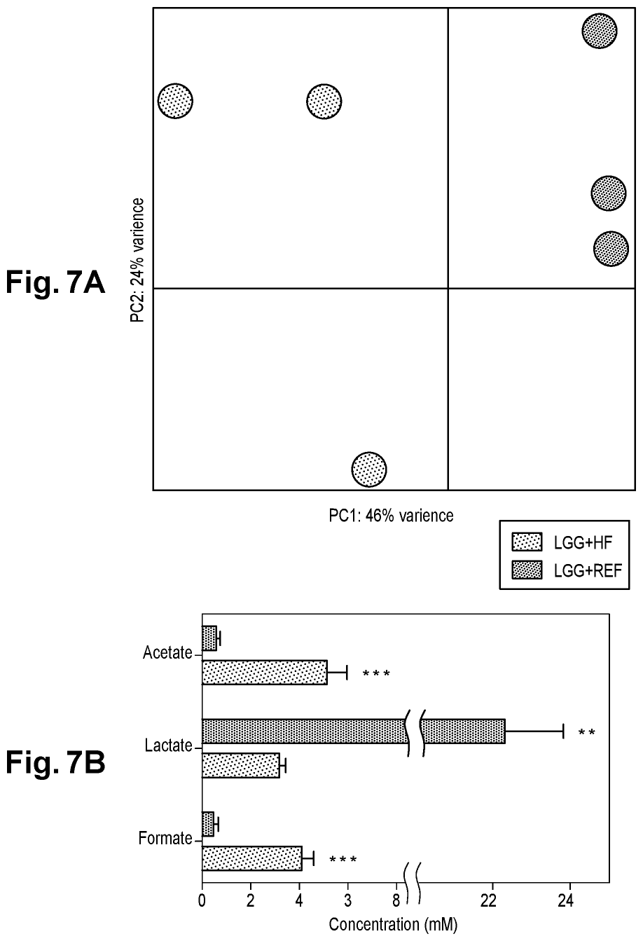

Even though LGG viability was not significantly affected by the presence of HF or REF medium (FIG. 6A), LGG growth was significantly reduced in the presence of the HF regimen when compared to REF (FIG. 6B).

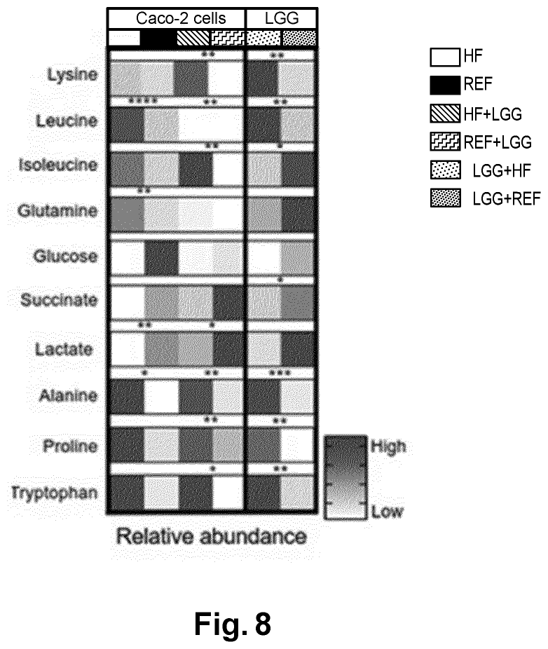

The simulated dietary regimen had a marked effect on the global transcriptome profile of LGG (FIG. 7A), similar to what has been observed for the human cells (356 differentially expressed genes, including 47 upregulated hypothetical proteins, in LGG when exposed to the simulated HF regimen in comparison to the REF medium). Genes encoding the cellobiose transporter were upregulated in LGG in the presence of the HF regimen, suggesting the catabolism of prebiotic components by LGG. Indeed, catabolism of prebiotic components used in the HF medium (e.g., arabinogalactan, xylan) has previously been suggested for Lactobacillus species (Douillard et al., 2013; Jaskari et al., 1998).

These prior observations were also actually supported by metabolomic analyses of organic and short-chain fatty acids in the supernatant after 48 hours of incubation of LGG in the HF medium. Significantly higher levels of acetate and formate, and less lactate were observed compared to when LGG was grown in the REF medium (FIG. 7B).

### Competition and Metabolic Cross Feeding Between the Probiotic and Caco-2 Cells:

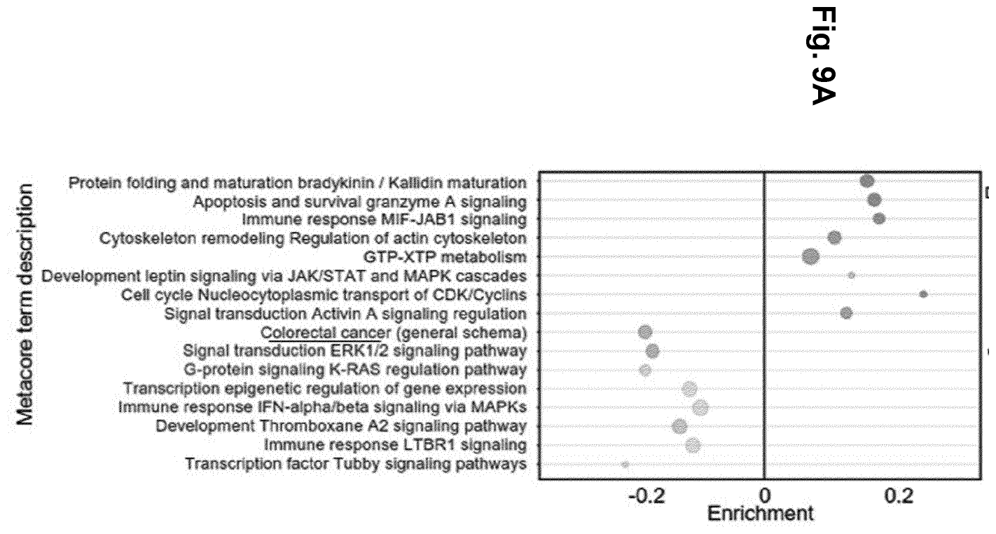

How the different dietary regimens tested affected the metabolism of the human Caco-2 cells and the probiotic LGG was tested by analyzing the intracellular metabolites of both cell contingents following co-culture in the HuMiX model (FIG. 8=5A). Although the intracellular concentrations of amino acids such as leucine and glutamine were higher in Caco-2 cells when grown in the presence of the HF regimen in comparison to cells grown in the presence of REF medium, the relative intracellular abundance of these amino acids in Caco-2 cells when co-cultured with the probiotic were significantly lower, regardless of the simulated diet used (FIG. 8).

In vitro measurements showed that relative intracellular concentrations of leucine and glutamine were higher in LGG when compared to Caco-2 cells (FIG. 8) indicating that the probiotic LGG outcompetes the host for these amino acids.

Similarly, the intracellular glucose concentrations in Caco-2 cells, which were highest when the cells were exposed to the REF medium alone, were significantly lower when Caco-2 cells were grown in the presence of the REF medium and LGG, suggesting that LGG was consuming the glucose, and thereby less glucose was available for the Caco-2 cells (FIG. 8).

By contrast, intracellular lactate concentrations were the highest in Caco-2 cells when exposed to the probiotic LGG, regardless of the diet used (FIG. 8). This result suggests therefore potential metabolic cross-feeding of lactate produced by the probiotic LGG.

### The Synbiotic Regimen Decreases Expression of Pro-Carcinogenic Genes and ABC-Transporters in Human Cells:

How the growth in the presence of the LGG probiotic altered gene expression in Caco-2 cells has been analyzed. Principal Component Analysis showed that the presence of LGG had an effect on the global transcriptome profile of human Caco-2 cells grown in the HF regimen but not when REF medium was used (FIG. 5A). A total of 1,771 genes were differentially expressed in Caco-2 cells grown in the simulated HF regimen in the presence of LGG compared to the expression in the same dietary regimen in the absence of LGG.

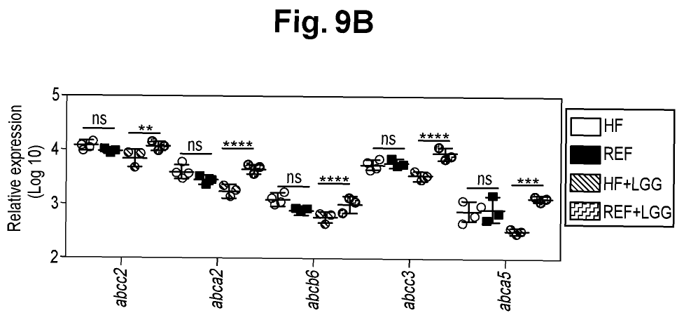

Pathway enrichment analysis showed that apoptosis and survival granzyme A signaling, as well as protein folding and maturation, were upregulated when Caco-2 cells were exposed to HF+LGG (synbiotic; FIG. 9A) but downregulated when exposed to HF alone.

Notably, a substantial number of CRC associated pathways were downregulated including the “colorectal cancer” pathway, and G-protein K-RAS signaling (FIG. 9A). Downstream targets of K-RAS signaling such as phosphatidylinositol 4,5-bisphosphate 3-kinase catalytic subunit alpha (PI3K-CA) were also downregulated only in the HF+LGG condition.

In addition to the downregulated CRC-associated pathways, the expression of several ABC transporters was significantly decreased in Caco-2 cells after co-culture with the combination of HF+LGG (FIG. 9B). ABC transporters have been implicated in drug resistance (Gottesman et al., 2002), and high abcc2 expression has also been associated with the early stages of CRC progression (Andersen et al., 2015). A search of the differentially expressed gene list of human Caco-2 cells grown in the presence of HF+LGG against the DrugBank database revealed that the downregulated genes abcc2, abcc3, cyp1a1, cox-2, and cyp2d6 all encode targets of CRC drugs. These results suggests that probiotics, dietary regimens and combinations thereof can affect major gene targets of CRC drugs.

### The Combination of Organic Ad Short Chain Fatty Acids Produced by LGG are Diet Dependent and Elicit Differential Effects in CRC Cells:

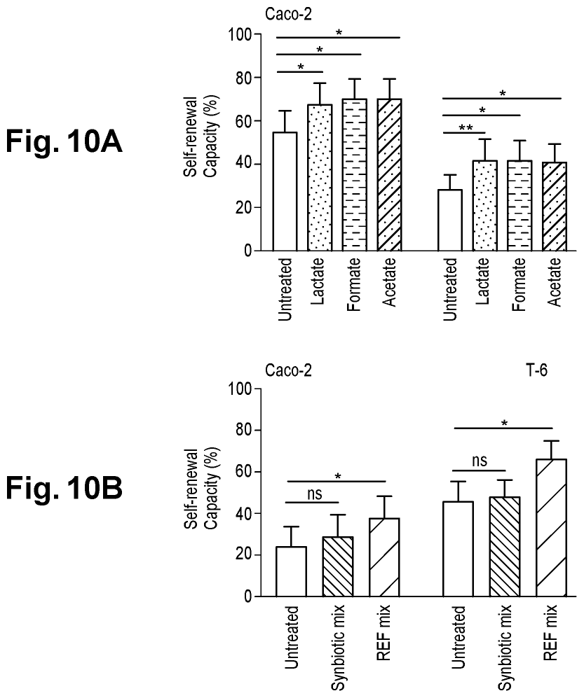

The observed changes in host gene expression could be due to the diet-dependent metabolites secreted by LGG (Thomas and Versalovic, 2010). As some of these pathways (e.g., PIK3-CA and the mammalian target of rapamycin (mTOR) signaling pathway) are related to cell self-renewal capacity (Xia and Xu, 2015), human Caco-2 cells and a primary CRC cell line (T-6) were stimulated with the fermentation products produced by the probiotic LGG in the presence of the HF or REF medium. The CRC cells were first separately exposed to 10 mM of the individual metabolites (which is between 2.5 and 12.5 times higher than the concentrations of the SCFAs produced by LGG). Under these conditions, the self-renewal capacity significantly increased in both Caco-2 and T-6 cells compared to the untreated controls (FIG. 10A). However, when the cells were treated with the respective ratios of metabolites produced by LGG when exposed to the two dietary regimens (Figure FIG. 7B), we observed that only the cocktail of molecules reflecting the synbiotic attenuated cancer cell self-renewal capacity. Thereby, the distinct ratios of organic and short-chain fatty acids produced by the probiotic are diet-dependent, and only the combination produced during the synbiotic regimen was able to revert the cellular hallmarks of tumor progression and disease dissemination.

### Material and Methods

### Simulated Dietary Regimens:

Two types of dietary regimens were used. The HF medium (prebiotic medium) is a modification from the simulated ileal environment medium (SIEM) (Gibson et al., 1988). SIEM medium contains 47 g/L bactopeptone (BD #211677), 78.4 g/L potato starch (Sigma #33615), 9.4 g/L xylan (from beechwood; Sigma #X4252), 9.4 g/L arabinogalactan (from larch wood; Sigma #10830), 9.4 g/L amylopectin (from maize; Sigma #10120), 9.4 g/L pectin (from apple; Sigma #76282), 3 g/L casein hydrosylate (Sigma #22090), 0.8 g/L dehydrated bile (Sigma #70168), and 4 g/L soy (Frutarom). All components were dissolved in distilled water with and heat (120° C.). The medium was autoclaved at 121° C., and 10 ml/L trace minerals (ATCC®MD-VS™), 10 ml/L Vitamin Supplement (ATCC®MD-TMS™), menadione (Sigma #M57405) and 100 mM cysteine HCl were filter-sterilized and added to the autoclaved medium. Menadione was dissolved in DMSO (1 mg/ml; D2650) prior to being added to the SIEM. After achieving complete homogeneity, the pH was adjusted to 7.0. The medium was conserved in aliquots at −20° C. until use. Before use, the medium was thawed at 37° C., and then transferred to a serum bottle with a rubber stopper and made anoxic. The reference (REF) medium (no-dietary-fiber medium) used was Dulbecco's Modified Eagle's medium (DMEM) (Sigma #6429) supplemented with fetal bovine serum (FBS) (Life Technologies). This REF medium provides the basic requirements for culture of both human Caco-2 cells and probiotic LGG (Shah et al., 2016).

### Human Cell Culture Conditions:

The human epithelial CRC cell line Caco-2 (DSMZ: ACC169) was maintained at 37° C. in a 5% CO₂ incubator in DMEM supplemented with 20% FBS. On day 1 of the HuMiX protocol, cells (at 6×10⁵ cells per mL) were injected using a sterile syringe into the epithelial chamber of HuMiX as described previously (Shah et al., 2016).

### Bacterial Growth Conditions:

Lactobacillus rhamnosus GG (LGG) (ATCC: 53103) cultures were started from glycerol stocks and precultured for 20 h in Brain Heart Infusion Broth (BHIS; Sigma #53286), supplemented with 1% hemin in an anaerobic chamber (Jacomex, TepsLabo Equipment, Dagneux, France) at 37° C., 5% CO₂ and <0.1% 02. After washing bacterial pellet with anoxic Phosphate Buffer Saline (PBS), LGG organisms were resuspended under anaerobic conditions in DMEM supplemented with 20% FBS; 1 mL of the microbial suspension (OD _{˜}1) was injected using a sterile syringe into the bacterial chamber of the device on day 7. For the experiments using the HF regimen, the bacterial chamber was primed on day 7 with the HF medium before the inoculation of bacteria.

The HuMiX model:

The assembly and setup steps of HuMiX have been described previously (Shah et al., 2016). In short, human Caco-2 cells (6×10⁵ cells per mL) were injected using a sterile syringe on day 1. After injection, the Caco-2 cells were allowed to attach to the collagen-coated microporous membrane at 37° C. Two hours of perfusion with anoxic and aerobic DMEM medium in a bacterial chamber and perfusion chamber, respectively, was carried out at a flow rate of 25 μL min⁻¹. On day 7, 1 mL of a bacterial suspension (OD _{˜}1) was injected into the bacterial chamber. The bacterial chamber was either perfused with the simulated HF medium or the REF medium. After a 24-hour co-culture, the HuMiX experiment was terminated, the device was disassembled, and LGG and human cells were collected from the distinct cell contingents as previously described (Shah et al., 2016). Two types of analyses were performed for each contingent whereby half of the biomass was used for biomolecular extraction, and a quarter was used for live/dead staining performed by flow cytometry (BD FACS Canto II, BD Biosciences San Jose USA).

### Biomolecular Extractions:

The detailed biomolecular extraction procedure performed on the HuMiX membrane-adherent human Caco-2 cells after 24-hour co-culture has been described previously (Shah et al., 2016). In short, Caco-2 cells were treated with a 1:1 methanol:water (v/v) solution, and polar and non-polar metabolite fractions were separated using chloroform. The Caco-2 interphase pellets were then processed using a Qiagen AllPrep DNA/RNA/Protein Mini Kit (Roume et al., 2013). The biomolecular extractions on the microbial cell contingents have been described previously (Shah et al., 2016). In short, the microbial cells were lysed using a Precellys lysis kit, and polar and non-polar metabolite fractions were obtained. The interphase pellet was processed using an All-in-One-Norgen Purification kit (Cat. No. 1024200) for the extraction of biomacromolecules (DNA, RNA and proteins).

### Intracellular Bacterial and Human Metabolite Extraction:

Polar and nonpolar phases, divided by an interphase, were formed using a 1:1 methanol:water (v/v) solution (for Caco-2 cells) and a 1:3 methanol:water (v/v) solution (for bacteria), and polar and non-polar metabolite fractions were separated using chloroform. The upper polar phase was transferred in duplicates into GC/MS glass vials (Chromatographie Zubehör Trott, Bovenden Germany) and dried overnight in a speed vac (LABCONCO, Kansas City, Mo.). The lower non-polar phase was also transferred into GC/MS glass vials in technical duplicates and dried overnight in the chemical hood. The remainder of the polar and non-polar phases was used for pooling. The bacterial interphase, including the milling beads, were snap-frozen and stored at −80° C. All GC-MS glass vials were capped and stored at −80° C. until GC-MS analyses.

### Statistical Analysis of Human and Bacterial Intracellular Metabolite Measurements:

Statistical significance was calculated using Welch's t-test (Welch, 1947). The Welch t-test provides more robustness to an analysis than the regular Student t-test, and thus, the Welch t-test is commonly applied in metabolomics datasets (Kogel et al., 2010; Theriot et al., 2014).

### Short-Chain Fatty Acid Extraction, Derivatization, and GC-MS Measurements:

The conditioned medium (cell-free supernatant containing soluble factors) was collected by centrifugation (4° C. for 10 min at 12,000×g) from 48-hour bacterial cultures in Hungate tubes (Glasgeratebau Ochs, Bovenden, Germany) using either the HF or REF medium. Briefly, 20 μl of the internal standard (2-Ethylbutyric acid, 20 mmol/L) were added to 180 μL of medium. After acidification with 10 μL of 37% hydrochloric acid, 1 mL of diethyl ether was added and the samples were vortexed for 15 min at 450×g at room temperature (Eppendorf Thermomixer). The upper organic phase was separated by centrifugation (5 min, 21,000×g) and 900 μL were collected in a new reaction tube. A further 1 mL of diethyl ether was then added to the medium, and the tube was incubated and its contents separated by centrifugation. Then, 900 μL of the organic phase were combined with the first extract, and 250 μL of this combined mixture were transferred into a GC glass vial with micro insert (5-250 μL), in triplicate. For derivatization, 25 μL of N-tert-Butyldimethylsilyl-Nmethyltrifluoroacetamide with 1% tert-Butyldimethylchlorosilane (MTBSTFA+1% TBDMSCI, Restek Bellefonte Pa.) was added, and the samples were incubated for a minimum of 1 h at room temperature. For quantification, an external calibration curve (10, 25, 50, 75, 100, 250, 500, 1000, 2000, 4000 μmol/L) using a volatile free acid mix (Sigma CRM46975 Supelco) including all compounds of interest was prepared, extracted, and derivatized as described previously (Moreau et al., 2003). Gas chromatography-mass spectrometry (GC-MS) analysis was performed using an Agilent 7890A GC coupled to an Agilent 5975C inert XL Mass Selective Detector (Agilent Technologies, Santa Clara Calif.). A sample volume of 1 μL was injected into a split/splitless inlet operating in split mode (20:1) at 270° C. The gas chromatograph was equipped with a 30 m (I.D. 250 μm, film 0.25 μm) DB-35MS capillary column (Agilent J&W GC Column). Helium was used as carrier gas, with a constant flow rate of 1.4 mL/min. The GC oven temperature was held at 80° C. for 1 min and increased to 150° C. at 10° C./min. Then, the temperature was increased to 280° C. at 50° C./min (post run time: 3 min). The total run time was 15 min. The transfer line temperature was set to 280° C. The mass selective detector was operating under electron ionization at 70 eV. The MS source was held at 230° C. and the quadrupole at 150° C. The detector was switched off during elution of MTBSTFA. For precise quantification, GC-MS measurements of the compounds of interest were performed in selected ion monitoring mode.

### Glucose and Lactate Measurements:

Glucose and lactate from the same conditioned medium from LGG culture in either simulated HF or REF medium, as described above, were measured using a YSI Biochemistry Analyzer (2950D, Yellow Springs, Ohio).

### Cell Viability and Counting:

After 24-hour HuMiX co-culture, LGG and human Caco2 cells were harvested from a quarter membrane for cell counting and staining. The mucin-coated bacterial membrane was first gently washed and resuspended in MACS buffer (PBS containing 1% BSA), then stained with PI/SYTO9 (Life Tech #L7012, Carlsbad, Calif.) and fixed with 4% PFA. Quantification of bacterial cells was performed by flow cytometry (BD FACS Canto II, BD Biosciences) using bacteria counting beads (Thermo Fischer B7277, Waltham Mass.) as a standard for the volume of suspension.

Human Caco-2 cells were stained with a near-IR fluorescent dye (Invitrogen L10119, Carlsbad, Calif.) and fixed with 4% PFA. The resulting data were analyzed using FlowJo software (BD Biosciences, version 10).

### Sphere and 3D Colony Formation Assays:

Self-renewal capacity was assessed with sphere formation assays, as previously described (Qureshi-Baig et al., 2016). Briefly, primary CRC cells T-6 and Caco-2 cells were seeded at different densities (e.g., 1, 2, or 3 cells per well), and after 10 days of culture, the resulting spheroids were counted and measured under a microscope. Extreme limiting dilution analysis software (Hu and Smyth, 2009) was used to determine the self-renewal capacity after a given treatment. 3D colony formation was assessed by resuspending the cells in serum-free medium and by seeding 250 cells (per 35 mm dish) in a mix of 60% SCM medium (QureshiBaig et al., 2016) and 40% methylcellulose medium, i.e., MethoCult® H4100 (STEMCELL Technologies, Vancouver, Canada), supplemented with EGF (20 ng/mL) (Biomol) and basic fibroblast growth factor (bFGF) (20 ng/mL) (Miltenyi Biotec, Bergisch Gladbach, Germany). The resulting colonies were counted after 14 days, using an inverted microscope.

### Primary CRC Cells T6:

Primary CRC tumor colon tissue was collected from an adenoma stage III CRC patient by the Integrated Biobank of Luxembourg (IBBL, www.ibbl.lu) in accordance with institutional guidelines and has previously been described (Ullmann et al., 2016).

### RNA Library Preparation for Caco-2 and LGG:

Sequencing library preparation was performed using a NEBNext, Ultra Directional RNA Library Prep Kit (Illumina E7420, San Diego Calif.) using 500 ng of total RNA isolated from LGG or Caco-2 cells cocultured inside HuMiX under the described media conditions. Briefly, for bacterial RNA samples, ribosomal RNA depletion was carried out using a Ribo-zero rRNA Removal Kit (Bacteria) (Illumina, San Diego Calif.) according to the manufacturer's protocol. Ribo-depleted RNA was purified using magnetic beads, resuspended into 5 μL of TE buffer and further processed for library preparation according to chapter 3 of the NEBNext, Ultra Directional RNA Library Prep Kit (Illumina E7420) protocol booklet.

The sequencing libraries for the Caco-2 RNA samples were prepared according to the protocol provided in chapter 2 of the NEBNext, Ultra Directional RNA Library Prep Kit (Illumina E7420). The libraries were quantified using a Qubit dsDNA HS Assay Kit (Thermo Fischer Scientific, Waltham Mass.), and quality was determined using an Agilent 2100 Bioanalyzer. Pooled libraries were sequenced on a NextSeq500 device using 2×75 cycle reaction chemistry. FASTQ file generation and demultiplexing were performed using bcl2fastq.

### RNAseq (Data Analysis):

To ensure complete removal of all rRNA, in silico rRNA depletion was performed using sortmeRNA (2.1) (Kopylova et al., 2012). The rRNA depletion was required only for the bacterial samples, as rRNA made up 85% of the total RNA (Rosenow et al., 2001; Scott et al., 2010), and thus, all other RNA classes would have been masked. For the human samples, rRNA depletion was not performed as only mRNAs were sequenced.

The remaining reads were mapped to the LGG reference genome (assembly ID: ASM2650v1) using bowtie2 (2.3.0) (Langmead and Salzberg, 2012) with a default setting at the very-sensitive-local mode. The reference genome was reannotated using eggnog-mapper based on eggNOG 4.5 orthology data (Huerta-Cepas et al., 2017), and gene counts were strand based, applying an in-house script. The DESeq2 (1.16.1) (Anders and Huber, 2010) package from R (3.4.1) (Team, 2016) was used to retrieve genes that were differentially expressed (DE) due to dietary regimen. DE genes with an absolute log 2-fold change value higher than 1 and an adjusted P value lower than 0.05 were tested for pathway and module enrichment using the R packages KEGGREST (Tenenbaum, 2017) and stats (Team, 2017) (hypergeometric distribution function). In a similar approach, Caco2 transcriptomic datasets were aligned against the Ensembl human genome reference (release-87) using the STAR (2.5.2b) aligner (Dobin et al., 2013) with default parameters, except for chimSegmentMin, which was set to 20 to switch on the detection of chimeric alignments. A GTF file (release-87) with the annotated transcripts was also provided to increase the accuracy of the alignments. Gene counts were calculated using feature Counts (v1.5.2) (Liao et al., 2014), requiring both ends to be mapped and strand specificity. To filter out genes that could be derived from spurious mapping, only genes that collected at least 0.0001% of the reads for a minimum of two samples from the same condition were kept. Generalized linear models were applied to calculate the differential gene expression and statistical significance for dietary regimen, type of culture (monoculture or coculture) and their interaction using DESeq2 (Anders and Huber, 2010).

Pathway enrichment analysis:

Pathway enrichment analysis on the Caco-2 differentially expressed gene list (HF regimen versus REF medium, and HF+LGG versus REF+LGG) was performed using MetaCore™ version 6.33 build 69110, using only the statistically significant genes as a sorting method.

1.-25. (canceled)

26. A method for use of a microfluidic cell culture device for performing dietary compounds—host microbiota cells molecular interactions, said microfluidic cells culture device comprising two or more channels;
wherein at least two adjacent channels are cell culture channels separated by a least one of a permeable and a semi permeable membrane adapted to prevent passage of cells thereacross;
wherein a first channel of the at least two adjacent channels supports a culture of microbiota cells of a host; and
wherein a second channel of the at least two channels supports at least one probiotics culture and is perfused with a medium of dietary compounds.

27. The method of use according to claim 26, for investigating combinatorial combinations of dietary compounds and probiotics, resulting in the production by probiotics of molecular compounds enabling to modulate at the molecular level the host microbiota cells.

28. The method of use according to claim 27, wherein dietary compounds comprise prebiotics.

29. The method of use according to claim 28, wherein prebiotics comprise dietary fibers, carbohydrates selected from the group consisting of disaccharides, oligosaccharides, polysaccharides, and/or mixtures thereof.

30. The method of use according to claim 29, wherein dietary fibers are selected from non-starch derived indigestible polysaccharides, galacto-oligosaccharides and fructo-oligosaccharides, and/or mixture thereof.

31. The method of use according to claim 27, wherein the molecular compounds secreted by the probiotics:
are dietary compound-dependent;
are prebiotics dependent;
comprise organic and short chain fatty acids; and/or
comprise lactate, formate, acetate.

32. The method of use according to claim 26, wherein the probiotics culture comprises at least one bacteria species.

33. The method of use according to claim 26, wherein the probiotics culture comprises at least one bacteria species from gut microbiome.

34. The method of use according to claim 26, wherein the probiotics culture comprises at least one bacteria species selected from Lactobacillus species.

35. The method of use according to claim 34, wherein the Lactobacillus species is selected from L. rhamnosus, L. acidophilus, L. delbrueckii, L. helveticus, L. casei, L. curvatus, L. plantarum, L. sakei, L. brevis, L. bruchneri, L. fermentum, L. reuteri.

36. The method of use according to claim 26, wherein the culture of microbiota cells and/or host microbiota cells is from a mammalian host.

37. The method of use according to claim 26, wherein the culture of microbiota cells and/or host microbiota cells is from a human host.

38. A synbiotic regimen obtained by using a microfluidic cells culture device for performing dietary compounds—host microbiota cells molecular interactions, said microfluidic cells culture device comprising two or more channels;
wherein at least two adjacent channels are cell culture channels separated by one of a permeable and a semi permeable membrane adapted to prevent passage of cells thereacross;
wherein a first channel of the at least two adjacent channels supports a culture of microbiota cells of a host; and
wherein a second channel of the at least two channels supports at least one probiotics culture and is perfused with a medium of dietary compounds.

39. The synbiotic regimen according to claim 38, for use in at least one of treating and preventing at least one of:
human colorectal cancer cells;
human gut microbiome-linked diseases; and
inflammatory diseases of gut.

40. The synbiotic regimen according to claim 38, for use as at least one of:
an adjuvant in combination with anti-cancer drug-treatments;
a dietary supplement in combination with anti-cancer drug treatments; and
a pharmaceutical composition.

41. The synbiotic regimen according to claim 38, which has the form of at least one of a liquid, a powder, a granulate, a paste, a bare, an effervescent tablet, a tablet, a capsule, a lozenge, one of a fast melting tablet and a wafer, and a substance tablet or a spray.

42. A method for performing dietary compounds—host microbiota cells molecular interactions comprising the following steps:
(i) providing a microfluidic device comprising two or more channels, at least two adjacent of the channels are cell culture channels separated by one of a permeable and a semi permeable membrane adapted to prevent passage of cells thereacross;
(ii) populating a first channel of the channels with a culture of gut cells from a host, the gut cells being selected from cells making up the wall in at least one of the small intestine, colon, and gastrointestinal tract epithelial cells;
(iii) passing a probiotics culture comprising at least one bacteria specie into a second channel of the channels;
(iv) perfusing through the second channel a medium of dietary compounds comprising prebiotics with dietary fiber;
(v) analyzing the interactions between said gut cells, prebiotics and probiotics by allowing the interrogation of molecular interactions by molecular techniques comprising at least one of a imaging, a spectroscopic technique and at least one of genomics, proteomics, metabolics, transcriptomics, and other molecular analysis techniques.

43. The method according to claim 42, wherein the culture of gut cells are one of mammalian, human or insect.
